(19) United States
(12) Patent Application Publication
(10) Pub. No.: US 2023/0340614 A1
(43) Pub. Date: Oct. 26, 2023

(54) TREATING TUMORS RESULTING IN OR FROM CNS METASTASIS USING MDM2/4 AND CDKN2A INHIBITORS

(71) Applicant: BROWN UNIVERSITY, Providence, RI (US)

(72) Inventors: Wafik S. El-Deiry, Barrington, RI (US); Taylor Arnoff, Providence, RI (US)

(21) Appl. No.: 18/139,402

(22) Filed: Apr. 26, 2023

Related U.S. Application Data
(60) Provisional application No. 63/334,762, filed on Apr. 26, 2022.

(51) Int. Cl. C12Q 1/6886; G01N 33/574

(52) U.S. Cl. CPC C12Q 1/6886; G01N 33/57484; C12Q 2600/106; C12Q 2600/156

(57) ABSTRACT

A method of treating tumors resulting in or from CNS metastasis in a subject is described. The method includes determining if the subject has an increased level of MDM2/MDM4 genes and/or proteins, and/or a decreased level of CDKN2A genes and/or proteins, and treating subjects having an increased level of MDM2/MDM4 genes and/or proteins with a combination of immunotherapy and an MDM2/MDM4 inhibitor, and/or treating subjects identified as having a decreased level of CDKN2A genes and/or proteins with a combination of immunotherapy and a CDK inhibitor.

### CROSS-REFERENCE TO RELATED APPLICATIONS

This application claims priority to U.S. Provisional patent application Ser. No. 63/334,762, filed Apr. 26, 2022, which is incorporated herein by reference.

### BACKGROUND

Brain metastases are the most common malignancy encountered in the central nervous system (CNS), with up to 30-40% of cancer patients developing brain metastases at some point during the course of their disease. Metastasis, involving the spread of systemic cancer to the brain, typically results in neurologic disability and death. Current treatments are largely palliative in nature; improved therapeutic approaches represent an unmet clinical need. Valiente et al., Trends Cancer, 4(3), 176-196 (2018).

The management of brain metastasis is rapidly evolving and the roles of local therapies such as whole-brain radiation therapy, stereotactic radiosurgery, and resection along with systemic therapies are in flux. Kotecha et al., F1000Res, 7:F1000 Faculty Rev-1772 (2018). However, melanoma currently has a five-year survival rate of 27% for distant metastatic disease. Immunotherapy is an active area of research in treating brain metastases. Drugs that target immune surface proteins CTLA4 (ipilimumab) and programmed cell death protein 1 (PD1) (pembrolizumab and nivolumab) have been developed and evaluated in patients with lung cancer and melanoma brain metastases. Berghoff et al., Am Soc Clin Oncol Educ Book, 35:e116-22 (2016). However, there remains a paucity of targeted therapies for metastatic disease and biomarkers that predict metastasis to specific sites, and in particular for metastases to the central nervous system.

### SUMMARY OF THE INVENTION

The present invention relates to a method of treating tumors resulting in or from CNS metastasis in a subject is described. The method includes determining if the subject has an increased level of MDM2/MDM4 genes and/or proteins, and/or a decreased level of CDKN2A genes and/or proteins, and treating subjects having an increased level of MDM2/MDM4 genes and/or proteins with a combination of immunotherapy and an MDM2/MDM4 inhibitor, and/or treating subjects identified as having a decreased level of CDKN2A genes and/or proteins with a combination of immunotherapy and a CDK inhibitor.

In some embodiments, the tumors result in CNS metastasis, such as metastatic melanoma. In some embodiments, the tumors result from CNS metastasis. For example, the tumors can be brain tumors, or in some embodiments, glioblastoma.

In some embodiments, the method includes determining the level of MDM2/MDM4 and/or CDKN2A genes in the subject. In other embodiments, the method includes determining the level of MDM2/MDM4 and/or CDKN2A proteins in the subject.

In some embodiments, the method includes determining if the subject has an increased level of MDM2 genes and/or proteins, and treating subjects having an increased level of MDM2 genes and/or proteins with a combination of immunotherapy an MDM2 inhibitor. In further embodiments, the method includes determining if the subject has an increased level of MDM4 genes and/or proteins, and treating subjects having an increased level of MDM4 genes and/or proteins with a combination of immunotherapy an MDM4 inhibitor. In yet further embodiments, the method includes determining if the subject has a decreased level of CDKN2A genes and/or proteins, and treating subjects identified as having a decreased level of CDKN2A genes and/or proteins with a combination of immunotherapy and a CDK inhibitor.

In some embodiments, the method further comprises the step of obtaining a biological sample from the subject, and analyzing the biological sample to determine if the subject has an increased level of MDM2/MDM4 genes and/or proteins, or a decreased level of CDKN2A genes and/or proteins.

Another aspect of the invention provides a method of treating tumors resulting in or from CNS metastasis in a subject, including determining if the subject has an increased level of MDM4 genes and/or proteins, and treating subjects having an increased level of MDM4 genes and/or proteins with a therapeutically effective amount of an MDM4 inhibitor. In some embodiments, the tumors are glioblastoma or metastatic melanoma.

An advantage of the present approach is the development of rational precision therapy for GBM and other solid tumors with brain metastases by targeting putative drivers associated with cancer spread to the brain.

### DETAILED DESCRIPTION OF THE INVENTION

### Definitions

As used herein, the term “diagnosis” can encompass determining the likelihood that a subject will develop a disease, or the existence or nature of disease in a subject. The term diagnosis, as used herein also encompasses determining the severity and probable outcome of disease or episode of disease or prospect of recovery, which is generally referred to as prognosis). “Diagnosis” can also encompass diagnosis in the context of rational therapy, in which the diagnosis guides therapy, including initial selection of therapy, modification of therapy (e.g., adjustment of dose or dosage regimen), and the like.

As used herein, the term “prognosis” refers to a prediction of the probable course and outcome of a disease, or the likelihood of recovery from a disease. Prognosis is distinguished from diagnosis in that it is generally already known that the subject has the disease, although prognosis and diagnosis can be carried out simultaneously. In the case of a prognosis for glioblastoma, the prognosis categorizes the relative severity of the glioblastoma, which can be used to guide selection of appropriate therapy for the glioblastoma.

As used herein, the terms “treatment,” “treating,” and the like, refer to obtaining a desired pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease.

Prevention or prophylaxis, as used herein, refers to preventing the disease or a symptom of a disease from occurring in a subject which may be predisposed to the disease but has not yet been diagnosed as having it (e.g., including diseases that may be associated with or caused by a primary disease). Prevention may include completely or partially preventing a disease or symptom.

The term therapy, as used herein, encompasses activity carried out to treat a disease. The specific activity carried out to conduct therapy can include use of surgery, radiotherapy, hormonal therapy, chemotherapy, or the use of one or more therapeutic agents (e.g., anticancer agents).

The terms “therapeutically effective” and “pharmacologically effective” are intended to qualify the amount of an agent which will achieve the goal of improvement in disease severity and the frequency of incidence over treatment of each agent by itself, while avoiding adverse side effects typically associated with alternative therapies. The effectiveness of treatment may be measured by evaluating a reduction in tumor load or decrease in tumor growth in a subject in response to the administration of anticancer agents. The reduction in tumor load may be represent a direct decrease in mass, or it may be measured in terms of tumor growth delay, which is calculated by subtracting the average time for control tumors to grow over to a certain volume from the time required for treated tumors to grow to the same volume.

Unless defined otherwise, all technical and scientific terms used herein have the same meaning as commonly understood by one of ordinary skill in the art to which this invention belongs.

As used herein and in the appended claims, the singular forms “a”, “and”, and “the” include plural referents unless the context clearly dictates otherwise. Thus, for example, reference to “a sample” also includes a plurality of such samples and reference to “the protein” includes reference to one or more protein molecules, and so forth. The term “and/or” refers to both or either of the indicated items.

Treating Tumors Resulting in or from CNS Metastasis

One aspect of the invention provides a method of treating tumors resulting in or from CNS metastasis in a subject, including determining if the subject has an increased level of MDM2/MDM4 genes and/or proteins, and/or a decreased level of CDKN2A genes and/or proteins, and treating subjects having an increased level of MDM2/MDM4 genes and/or proteins with a combination of immunotherapy and an MDM2/MDM4 inhibitor, and treating subjects identified as having a decreased level of CDKN2A genes and/or proteins with a combination of immunotherapy and a CDK inhibitor. MDM2, MDM4 and CDKN2A are sometimes referred to herein as the biomarkers. Note that the use of the forward slash mark corresponds to “and or” with regard to MDM inhibitors when used herein.

In some embodiments, the method includes determining if the subject has an increased level of MDM2, and treating subjects having an increased level of MDM2 with a combination of immunotherapy an MDM2 inhibitor. In further embodiments, the method includes determining if the subject has an increased level of MDM4, and treating subjects having an increased level of MDM4 with a combination of immunotherapy an MDM4 inhibitor. In yet further embodiments, the method includes determining if the subject has a decreased level of CDKN2A, and treating subjects identified as having a decreased level of CDKN2A with a combination of immunotherapy and cyclin-dependent kinase (CDK) inhibitor.

Murine Double Minute 2 homolog (MDM2) is an E3 ubiquitin ligase and negative regulator of the tumor suppressor protein p53. Under normal conditions, MDM2 is constitutively spliced to generate a full-length protein, which self-dimerizes and promotes the proteasome-mediated degradation of p53. Momand et al., Cell, 69, 1237-1245 (1992). MDM2 is known to being an oncogene. Oliner et al., Cold Spring Harb Perspect Med, 6(6):a026336 (2016). The full-length transcript of the mdm2 gene encodes a protein of 491 amino acids with a predicted molecular weight of 56 kDa. This protein contains several conserved structural domains including an N-terminal p53 interaction domain, the structure of which has been solved using x-ray crystallography. In some embodiments, the MDM2 is human MDM2, which is also known as Hdm2.

Protein Mdm4 is a protein that in humans is encoded by the Mouse double minute 4 (MDM4) gene, which is also known as MDMX. Haupt et al., J Mol Cell Biol., 11(3):231-244 (2019). The human MDM4 gene, which plays a role in apoptosis, encodes a 490-amino acid protein containing a RING finger domain and a putative nuclear localization signal. The MDM4 putative nuclear localization signal, which all Mdm proteins contain, is located in the C-terminal region of the protein. The mRNA is expressed at a high level in thymus and at lower levels in all other tissues tested. MDM4 protein produced by in vitro translation interacts with p53 via a binding domain located in the N-terminal region of the MDM4 protein. MDM4 shows significant structural similarity (˜55%) to p53-binding protein MDM2. Mdm4 can act alone or together with mdm2 to inhibit and/or degrade p53.

CDKN2A, also known as cyclin-dependent kinase inhibitor 2A, is a gene which in humans is located at chromosome 9, band p21.3. It is ubiquitously expressed in many tissues and cell types. The gene codes for two proteins, including the INK4 family member p16 (or p16INK4a) and p14 arf, both of which act as tumor suppressors. The term CDKN2A protein, as used herein, refers to p16 and/or p14 arf. Germline mutations of CDKN2A are associated with familial melanoma, glioblastoma and pancreatic cancer. “Genetics of Skin Cancer”. National Cancer Institute. Jul. 29, 2009.

As used herein, the terms “tumor” or “cancer” refer to a condition characterized by anomalous rapid proliferation of abnormal cells of a subject. The abnormal cells often are referred to as “neoplastic cells,” which are transformed cells that can form a solid tumor. The term “tumor” refers to an abnormal mass or population of cells (e.g., two or more cells) that result from excessive or abnormal cell division, whether malignant or benign, and pre-cancerous and cancerous cells. Malignant tumors are distinguished from benign growths or tumors in that, in addition to uncontrolled cellular proliferation, they can invade surrounding tissues and can metastasize.

The method includes treating tumors resulting in or from CNS metastasis. In some embodiments, the tumors result in CNS metastasis. Tumors resulting in CNS metastasis are tumors that are identified as being capable or having a higher likelihood of metastasizing into the central nervous system (CNS). For example, metastatic melanoma may be capable of metastasizing into the central nervous system. Such tumors are generally known as primary tumors. The most common types of cancer that can spread to the CNS are lung, breast, skin (i.e., melanoma), colon, kidney, and thyroid cancer.

In some embodiments, the tumors result from CNS metastasis. A tumor resulting from CNS metastasis is a tumor resulting from the spread of cancer from a primary tumor to a secondary site, and in this case, a secondary site in the CNS. Such tumors are referred to as secondary tumors. In some embodiments, the tumor resulting from CNS metastasis is a brain tumor. Metastatic brain tumors are five times more common than primary tumors in the brain. In further embodiments, the tumor resulting from CNS metastasis is a glioblastoma.

Another aspect of the invention provides a method of treating tumors resulting in or from CNS metastasis in a subject, including determining if the subject has an increased level of MDM4, and treating subjects having an increased level of MDM4 with a therapeutically effective amount of an MDM4 inhibitor. In this embodiment, the MDM4 inhibitor can be administered alone, without being used in combination with an immunotherapeutic agent. In some embodiments, the tumors resulting in or from CNS metastasis are glioblastoma or metastatic melanoma.

### Biological Samples and Subjects

In some embodiments, the method further comprising the step of obtaining a biological sample from the subject and analyzing the biological sample to determine if the subject has an increased level of MDM2/MDM4 genes and/or proteins, and/or a decreased level of CDKN2A genes and/or proteins. An increased or decreased level refers to a higher or lower level, respectively, of the indicated gene or protein, as compared with the control level values found in average or healthy individuals. The increased or decreased level can be at least a 5%, 10%, 20%, 30%, 40%, 50%, 60%, 70%, 80%, 90%, or at least 100% change relative to the control value.

A “biological sample,” as used herein, is meant to include any biological sample from a subject that is suitable for analysis for detection of the of MDM2, MDM4, CDKN2A genes or their associated RNA or proteins. Suitable biological samples include but are not limited to bodily fluids such as blood-related samples (e.g., whole blood, serum, plasma, and other blood-derived samples), urine, sputum, cerebral spinal fluid, bronchoalveolar lavage, and the like. Another example of a biological sample is a tissue sample. In some embodiments, the biological sample is a cancer cell or tissue including cancer cells. The biomarkers can be assessed either quantitatively or qualitatively, and detection can be determined either in vitro or ex vivo.

The methods involve providing or obtaining a biological sample from the subject, which can be obtained by any known means including needle stick, needle biopsy, swab, and the like. In an exemplary method, the biological sample is a blood sample, which may be obtained for example by venipuncture.

A biological sample may be fresh or stored. Biological samples may be or have been stored or banked under suitable tissue storage conditions. The biological sample may be a tissue sample expressly obtained for the assays of this invention or a tissue sample obtained for another purpose which can be subsampled for the assays of this invention. Preferably, biological samples are either chilled or frozen shortly after collection if they are being stored to prevent deterioration of the sample.

The sample may be pretreated as necessary by dilution in an appropriate buffer solution, heparinized, concentrated if desired, or fractionated by any number of methods including but not limited to ultracentrifugation, fractionation by fast performance liquid chromatography (FPLC) or HPLC, or precipitation of apolipoprotein B containing proteins with dextran sulfate or other methods. Any of a number of standard aqueous buffer solutions at physiological pH, such as phosphate, Tris, or the like, can be used.

The terms “individual,” “subject,” and “patient” are used interchangeably herein irrespective of whether the subject has or is currently undergoing any form of treatment. As used herein, the term “subject” generally refers to any vertebrate, including, but not limited to a mammal Examples of mammals including primates, including simians and humans, equines (e.g., horses), canines (e.g., dogs), felines, various domesticated livestock (e.g., ungulates, such as swine, pigs, goats, sheep, and the like), as well as domesticated pets (e.g., cats, hamsters, mice, and guinea pigs). Treatment or diagnosis of humans is of particular interest.

### Methods for Detecting MDM2/MDM4, or CDKN2A

In some embodiments, the method of treating tumors resulting in or from CNS metastasis includes determining the levels of the biomarkers MDM2/MDM4, and/or CDKN2A. Methods for determining the levels of the biomarkers MDM2/MDM4, and/or CDKN2A differ depending on whether the gene or the protein is being detected. The gene or protein can be detected or measured by an analytic device such as a kit or a conventional laboratory apparatus, which can be either portable or stationary. In some embodiments, the levels of variant gene or protein may be compared to the level of corresponding internal standards in the sample or samples when carrying out the analysis to quantify the amount of the gene or protein being detected.

In some embodiments, the level of a biomarker protein is determined. The level of a biomarker protein in a biological sample can be determined using polyclonal or monoclonal antibodies that are immunoreactive with the biomarker protein. Use of antibodies comprises contacting a sample taken from the individual with one or more of the antibodies; and assaying for the formation of a complex between the antibody and a protein or peptide in the sample. For ease of detection, the antibody can be attached to a substrate such as a column, plastic dish, matrix, or membrane, preferably nitrocellulose. The sample may be untreated, subjected to precipitation, fractionation, separation, or purification before combining with the antibody. Interactions between antibodies in the sample and the biomarker protein are detected by radiometric, colorimetric, or fluorometric means, size-separation, or precipitation. Preferably, detection of the antibody-protein or peptide complex is by addition of a secondary antibody that is coupled to a detectable tag, such as for example, an enzyme, fluorophore, or chromophore. Formation of the complex is indicative of the presence of the biomarker protein in the sample.

Antibodies immunospecific for the biomarker protein may be made and labeled using standard procedures and then employed in immunoassays to detect the presence of the biomarker in a sample. Suitable immunoassays include, by way of example, immunoprecipitation, particle immunoassay, immunonephelometry, radioimmunoassay (RIA), enzyme immunoassay (EIA) including enzyme-linked immunosorbent assay (ELISA), sandwich, direct, indirect, or competitive ELISA assays, enzyme-linked immunospot assays (ELISPOT), fluorescent immunoassay (FIA), chemiluminescent immunoassay, flow cytometry assays, immunohistochemistry, Western blot, and protein-chip assays using for example antibodies, antibody fragments, receptors, ligands, or other agents binding the target analyte. Polyclonal or monoclonal antibodies raised against the biomarker protein are produced according to established procedures. Generally, for the preparation of polyclonal antibodies, a protein or peptide fragment thereof is used as an initial step to immunize a host animal. A general review of immunoassays is available in Methods in Cell Biology v. 37: Antibodies in Cell Biology, Asai, ed. Academic Press, Inc. New York (1993), and Basic and Clinical Immunology 7th Ed., Stites & Terr, eds. (1991).

In some embodiments, the biomarker protein is detected using a method other than an immunoassay. For example, the biomarker protein can be detected using matrix-assisted laser desorption-ionization time-of-flight mass spectrometry (MALDI-TOF). The biomarker protein can also be detected by purifying the protein and determining its sequence using peptide sequencing methods. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified and/or quantified using chromatographic and electrophoretic techniques to achieve partial or complete purification (or purification to homogeneity). Analytical methods particularly suited to the preparation of a pure peptide are immunohistochemistry, ion-exchange chromatography, exclusion chromatography; polyacrylamide gel electrophoresis; isoelectric focusing. A particularly efficient method of purifying peptides is fast protein liquid chromatography or even HPLC. Likewise, a variety of methods of protein sequencing are known to those skilled in the art. For example, the sequence may be identified using mass spectrometry or the Edman degradation reaction.

In some embodiments, the presence of the biomarker gene is determined. The presence and/or the level of the biomarker gene can be determined by any now known or hereafter developed assay or method of detecting and/or determining expression level, for example, quantitative RT-PCR, Northern blot, real-time PCR, PCR, allele-specific PCR, pyrosequencing, SNP Chip technology, or restriction fragment length polymorphism (RFLP).

Many of the methods for determining a nucleotide sequence involve PCR. As used herein, the term “polymerase chain reaction” (PCR) refers to the methods of U.S. Pat. Nos. 4,683,195, 4,683,202, and 4,965,188, all of which are hereby incorporated by reference, directed to methods for increasing the concentration of a segment of a target sequence in a mixture of genomic DNA without cloning or purification. As used herein, the terms “PCR product” and “amplification product” refer to the resultant mixture of compounds after two or more cycles of the PCR steps of denaturation, annealing and extension are complete. These terms encompass the case where there has been amplification of one or more segments of one or more target sequences. Accordingly, in some embodiments, the detecting the presence and/or level of the biomarker gene comprises extending a primer that hybridizes to a sequence adjacent to the polymorphic nucleotide. In some embodiments, the determining the presence and/or level of the biomarker gene comprises hybridizing a probe to a region that includes the polymorphic nucleotide.

In some embodiments, hybridization with complementary sequences may be used to detect the presence of the biomarker gene based on the different characteristics of sequences that have a complete or incomplete sequence match. For example, as described herein, an asymmetric PCR assay can be used in which fluorescence melting reveals two distinct melting temperatures of the probe/target duplex that are specific for the amplified biomarker gene. This assay can be used to detect the biomarker gene in the germline or in somatic cells.

Once the presence and/or levels of the biomarker have been determined, they can be displayed in a variety of ways. For example, the levels can be displayed graphically on a display as numeric values or proportional bars (i.e., a bar graph) or any other display method known to those skilled in the art. The graphic display can provide a visual representation of the amount of the gene, RNA, or protein in the biological sample being evaluated.

### Therapeutic Methods

The present invention includes treating subjects having an increased level of MDM2/MDM4 with a combination of immunotherapy and an MDM2/MDM4 inhibitor, and treating subjects identified as having a decreased level of CDKN2A with a combination of immunotherapy and a cyclin-dependent kinase (CDK).

Immunotherapy, and in particular cancer immunotherapy, is the modulation (e.g., stimulation) of the immune system to treat cancer by improving on the immune system's natural ability to fight the disease. Examples of cancer immunotherapy include cellular immunotherapy (e.g., dendritic cell therapy, CAR-T cell therapy, and T cell receptor T cell therapy), antibody therapy (e.g., administration of nivolumab, pembrolizumab, atezolizumab, durvalab, ipilimumab, or avelumab), and cytokine therapy (e.g., administration of an interferon or interleukin). In some embodiments, the cancer immunotherapy comprises immune checkpoint inhibitor therapy. There are currently six FDA-approved immunotherapy options for brain and nervous system cancers. These include dostarlimab, GM-CSF, pembrolizumab, bevacizumab, dinutuximab, and naxitamab-gqgk.

Immune checkpoint inhibitor therapy blocks immune checkpoints affecting immune system function. Tumors can use these checkpoints to protect themselves from immune system attacks. Blockade of negative feedback signaling to immune cells thus results in an enhanced immune response against tumors. Examples of immune checkpoint inhibitor therapy includes CTLA-4 blockage, administration of PD-1 inhibitors, administration of PD-L1 inhibitors, intrinsic checkpoint blockade (CISH), and administration of oncolytic viruses.

In some embodiments, an MDM2 inhibitor is used for treatment of the subject. A variety of MDM2 inhibitors are known to those skilled in the art. See Qin et al., Curr Med Chem.; 19(33): 5705-5725 (2012), Zhu et al, J Hematol Oncol., 15: 91 (2022), and Khoury K., Dömling A., Curr Pharm Des, 18(30):4668-78 (2012), the disclosures of which are incorporated herein by reference. Examples of MDM2 inhibitors include Nutlin-3, the spirooxindoles M143, MI-83, and MI219, RG7112, RG7368, MK-8242, AMG232, SARf-5838/MI77301, DS-3032b, HDM201, NVP-GCM097, and APG115.

In some embodiments, an MDM4 inhibitor is used for treatment of the subject. A variety of MDM4 inhibitors are known to those skilled in the art. See Woodfield et al., Sci Rep., 11(1):2967 (2021), Zhang et al., J. Med. Chem., 64, 15, 10621-10640 (2021), and Markey M., Front Biosci (Landmark Ed), 16(3):1144-56 (2011), the disclosures of which are incorporated by reference herein. Examples of MDM4 inhibitors include ALRN-6924, ATPS-7041, and NSC207895 (XI-006).

In some embodiments, both an MDM2 inhibitor and an MDM4 inhibitor are used. Some references teach examples of both of these types of inhibitors. See Miles et al., Front Oncol, 11:703442 (2021), Toledo F, Wahl G., Int J Biochem Cell Biol. 2007; 39(7-8):1476-82. (2007), and Duffy et al., Semin Cancer Biol, 79:58-67 (2022), the disclosures of which are incorporated herein by reference. In some cases, a single molecule such as lithocholic acid can be used to inhibit both MDM2 and MDM4. Vogel et al., Proc Natl Acad Sci USA, 109(42): 16906-10 (2012).

In some embodiments, a cyclin-dependent kinase (CDK) inhibitor is used for treatment of the subject. Cyclin-dependent kinases are a family of protein kinases involved in regulation of the cell cycle. CDKs are relatively small proteins, with molecular weights ranging from 34 to 40 kDa, and contain little more than the kinase domain. Examples of cyclin-dependent kinases include Cdk1, Cdk2, Cdk3, Cdk4, Cdk5, Cdk6, Cdk7, Cdk8, and Cdk9. A wide variety of cyclin-dependent inhibitors are known to those skilled in the art. See Ettl et al., Cancers (Basel), 14(2):293 (2022), Juric V., Murphy B., Cancer Drug Resist., 3(1):48-62 (2020), and Mariaule G., Belmont P., Molecules, 19(9):14366-82 (2014), the disclosures of which are incorporated herein by reference. Examples of cyclin-dependent kinase inhibitors include flavopiridol, R-roscovitine, Dinaciclib, AT7519, SNS032, Palbociclib, EM-1421, RGB-286638, P276-00, BAY 1000394, TG02 (SB1317), PHA-848125, LEE-011, and Bemaciclib. In some embodiments, the CDK inhibitor is a CDK2 inhibitor. Examples of CDK2 inhibitors include flavopiridol, R547, NU2058, P276-00, AT7519, and Roscovitine. See Li et al., Int J Mol Sci., 16(5):9314-40 (2015), the disclosure of which is incorporated herein by reference.

Candidate agents may be tested in animal models. Typically, the animal model is one for the study of cancer. The study of various cancers in animal models (for instance, mice) is a commonly accepted practice for the study of human cancers. For instance, the nude mouse model, where human tumor cells are injected into the animal, is commonly accepted as a general model useful for the study of a wide variety of cancers, including prostate cancer (see, for instance, Polin et al., Investig. New Drugs, 15:99-108 (1997)). Results are typically compared between control animals treated with candidate agents and the control littermates that did not receive treatment. Transgenic animal models are also available and are commonly accepted as models for human disease (see, for instance, Greenberg et al., Proc. Natl. Acad. Sci. USA, 92:3439-3443 (1995)). Candidate agents can be used in these animal models to determine if a candidate agent decreases one or more of the symptoms associated with the cancer, including, for instance, cancer metastasis, cancer cell motility, cancer cell invasiveness, or combinations thereof.

The inventors analyzed 1081 primary melanoma samples and 358 metastatic melanoma samples and found that metastatic disease is enriched for amplifications in both MDM2 and MDM4 compared to primary disease, and these amplifications are associated with a lower probability of overall survival. Two additional negative regulators of TP53, namely USP7 and PPM1D, are enriched for alterations in metastatic melanoma compared to primary melanoma. MDM4 amplifications are associated with a higher rate of metastasis to the brain, liver, and lungs, while MDM2 amplifications are associated with a higher rate of metastasis to the brain, liver, and adrenal glands. These findings suggest that patients with metastatic melanoma show an enhanced dysregulation of the TP53 pathway compared to primary disease; though still under ongoing preclinical evaluation to assess therapeutic implications, the inventors propose that patients with metastatic melanoma and TP53 wild-type status may be more likely to benefit from mouse double minute 2 (MDM2), mouse double minute 4 (MDM4), USP7, and PPM1D inhibitors, both alone and in combination, compared to those with primary disease.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebral spinal, and intrasternal injection and infusion. In preferred embodiments, the compositions are administered by intravenous infusion or injection.

The therapeutic agents described herein can be supplied in the form of a pharmaceutical composition, comprising an isotonic excipient prepared under sufficiently sterile conditions for human administration. The composition can be sterile. The formulation should suit the mode of administration. Choice of excipient and any accompanying elements of the composition comprising a therapeutic agent will be adapted in accordance with the route and device used for administration. In some embodiments, the therapeutic agents can be administered as nanotherapeutics. Song et al., Trends Cancer, 6(4):288-298 (2020).

In some embodiments, the therapeutic agents are administered together with a pharmaceutically acceptable carrier. Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the therapeutic agents.

An example has been included to more clearly describe particular embodiments of the invention. However, there are a wide variety of other embodiments within the scope of the present invention, which should not be limited to the particular example provided herein.

### Example

The inventors found that patients with MDM2 alterations were more likely to have a deep deletion in cyclin dependent kinase inhibitor 2A (CDKN2A), alterations that are also associated with a higher rate of metastasis to the brain. They found that patients with a CDKN2A deep deletion had a statistically significant higher rate of alterations in TTN, MUC16, LRP1B, NF1, and SERPINB4, alterations that have all been previously associated with a favorable response to immune checkpoint inhibitors in melanoma. The inventors therefore propose that CDKN2A deletion may serve as a biomarker to predict response to immunotherapy in melanoma. Moreover, given prior documented cases of patients diagnosed with both melanoma and glioblastoma multiforme (GBM), the inventors found that GBM displays the highest rate of deep deletions in CDKN2A (54.39%) across all cancer types screened. They analyzed 619 GBM samples and found that 9.16% display an MDM2 amplification and 9.52% display an MDM4 amplification. Given the genomic similarities between melanoma and glioblastoma, they suggest that patients with melanoma or GBM and amplifications in MDM2/4 and CDKN2A deletions may need the development of combinations of targeted inhibitors of MDM2/4, CDK's and immunotherapy.

At this juncture, given similarities between melanoma or other tumors with brain metastases and glioblastoma, it appears that patients with amplifications in MDM2/4 and CDKN2A deletions may need the development of combinations of targeted inhibitors of MDM2/4, CDK's and immunotherapy. Some patients will have MDM2 amplification while others MDM4 or MDMX amplifications that will benefit from targeted inhibitors of these proteins −/+ immune checkpoint blockade, while others with CDKN2A mutation or deletion are predicted to respond better to immune checkpoint blockade.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of treating tumors resulting in or from CNS metastasis in a subject, including determining if the subject has an increased level of MDM2/MDM4 genes and/or proteins, and/or a decreased level of CDKN2A genes and/or proteins, and treating subjects having an increased level of MDM2/MDM4 genes or proteins with a combination of immunotherapy an MDM2/MDM4 inhibitor, and treating subjects identified as having a decreased level of CDKN2A genes or proteins with a combination of immunotherapy and a CDK inhibitor.

2. The method of claim 1, wherein the tumors result in CNS metastasis.

3. The method of claim 2, wherein the tumors are metastatic melanoma.

4. The method of claim 1, wherein the tumors result from CNS metastasis.

5. The method of claim 1, wherein the tumors are brain tumors.

6. The method of claim 4, wherein the tumors are glioblastoma.

7. The method of claim 1, wherein the method includes determining the level of MDM2/MDM4 and/or CDKN2A genes in the subject.

8. The method of claim 1, wherein the method includes determining the level of MDM2/MDM4 and/or CDKN2A proteins in the subject.

9. The method of claim 1, wherein the method includes determining if the subject has an increased level of MDM2 genes and/or proteins, and treating subjects having an increased level of MDM2 genes and/or proteins with a combination of immunotherapy an MDM2 inhibitor.

10. The method of claim 1, wherein the method includes determining if the subject has an increased level of MDM4 genes and/or proteins, and treating subjects having an increased level of MDM4 genes and/or proteins with a combination of immunotherapy an MDM4 inhibitor.

11. The method of claim 1, wherein the method includes determining if the subject has a decreased level of CDKN2A genes and/or proteins, and treating subjects identified as having a decreased level of CDKN2A genes and/or proteins with a combination of immunotherapy and a CDK inhibitor.

12. The method of claim 1, further comprising the step of obtaining a biological sample from the subject, and analyzing the biological sample to determine if the subject has an increased level of MDM2/MDM4 genes and/or proteins, or a decreased level of CDKN2A genes and/or proteins.

13. The method of claim 1, wherein the subject is human.

14. A method of treating tumors resulting in or from CNS metastasis in a subject, including determining if the subject has an increased level of MDM4 genes and/or proteins, and treating subjects having an increased level of MDM4 genes and/or proteins with a therapeutically effective amount of an MDM4 inhibitor.

15. The method of claim 14, wherein the tumors are glioblastoma or metastatic melanoma.
